(19) United States
(12) Patent Application Publication
(10) Pub. No.: US 2012/0115900 A1
(43) Pub. Date: May 10, 2012

(54) SUBSTITUTED NAPHTHYRIDINE DERIVATIVES AND THEIR MEDICAL USE

(75) Inventors: William Dalby Brown, Soborg (DK); Carsten Jessen, Birkerod (DK); Cecilia Mattsson, Frolunda (SE); Richard Sott, Molndal (SE); Dorte Strøbæk, Farum (DK)

(73) Assignee: NEUROSEARCH A/S, Ballerup (DK)

(21) Appl. No.: 13/265,273

(22) PCT Filed: Apr. 21, 2010
(86) PCT No.: PCT/EP2010/055284

§ 371 Date: Dec. 9, 2011

Related U.S. Application Data
(60) Provisional application No. 61/172,822, filed on Apr. 27, 2009.

(30) Foreign Application Priority Data
Apr. 21, 2009 (DK) PA 2009 00516

(51) Int. Cl. A61K 31/444; A61K 31/4545; A61P 25/08; A61P 25/04; A61P 25/18; A61P 25/00; A61P 25/22; A61P 25/24; C07D 471/04; A61P 25/06

(52) U.S. Cl. 514/300; 546/122

(57) ABSTRACT

The present application discloses novel substituted naphthyridine derivatives of Formula (I) and their use as modulators of the voltage gated Kᵥ7 (KCNQ) potassium ion channels. In other aspects the application discloses the use of these compounds, in a method for therapy and to pharmaceutical compositions comprising these compounds.

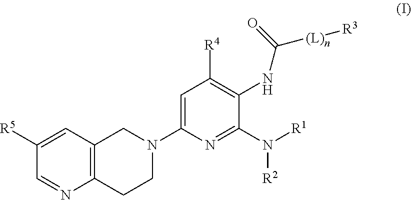

### TECHNICAL FIELD

The present invention relates to novel substituted naphthyridine derivatives, to their use in therapy, to pharmaceutical compositions comprising the derivatives, to the use of said derivatives in the manufacture of a medicament, and to therapeutic methods comprising the administration of the derivatives. The present derivatives are useful for treating a disorder, disease or a condition of a subject, which disorder, disease or condition is responsive to activation of Kᵥ7 channels.

### BACKGROUND ART

Potassium (K⁺) channels are structurally and functionally diverse families of K⁺-selective channel proteins, which are ubiquitous in cells, indicating their central importance in regulating a number of key cell functions. While widely distributed as a class, K⁺ channels are differentially distributed as individual members of this class or as families.

Recently a new family of potassium channels, the KCNQ channels, now also designated Kᵥ7, of which Kᵥ7.1-Kᵥ7.5 have currently been characterised, has attracted attention as target for therapeutic development.

Due to the distribution of Kᵥ7 channels within the organism, Kᵥ7 channel modulators are considered potentially useful for the treatment or alleviation of conditions as diverse as CNS disorders, psychiatric disorders, CNS damage caused by trauma, stroke or neurodegenerative illness or diseases, a variety of neuronal hyperexcitability disorders and conditions, epilepsy, pain, neuropathic pain, migraine, tension type headache, learning and cognitive disorders, motion and motor disorders, multiple sclerosis, cardiac disorders, heart failure, cardiomyopathia, inflammatory diseases, ophthalmic conditions, deafness, progressive hearing loss, tinnitus, obstructive or inflammatory airway diseases, for inducing or maintaining bladder control including the treatment or prevention of urinary incontinence.

WO2009/018466 (Valeant Pharmaceuticals International) discloses naphthyridine derivatives as potassium channel modulators. However this document does no disclose or suggest any cyclic amine substituents in the 2-position. WO2008/157404 (Valeant Pharmaceuticals International) discloses 4-(Nazacycloalkyl)anilide derivatives as potassium channel modulators. Also, this document does no disclose or suggest any cyclic amine substituents in the 2-position.

### SUMMARY OF THE INVENTION

The present invention discloses novel substituted naphthyridine compounds having medical utility for combating disorders, diseases or conditions responsive to activation of Kᵥ7 channels.

In one embodiment the present invention provides compounds of Formula (I)

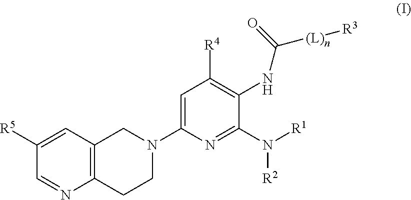

a stereoisomer or a mixture of its stereoisomers, or a pharmaceutically-acceptable addition salt thereof, or an N-oxide thereof, wherein R¹, R², R³, R⁴, R⁵, L and n are as defined below.

In another embodiment the invention provides pharmaceutical compositions comprising a therapeutically effective amount of a compound of the invention, a stereoisomer or a mixture of its stereoisomers, or a pharmaceutically-acceptable addition salt thereof, or an N-oxide thereof or a pharmaceutically-acceptable addition salt thereof.

In another embodiment the invention relates to the use of a compound of the invention, a stereoisomer or a mixture of its stereoisomers, or a pharmaceutically-acceptable addition salt thereof, or an N-oxide thereof, for the manufacture of pharmaceutical compositions.

In another embodiment the invention relates to the use of a compound of the invention, a stereoisomer or a mixture of its stereoisomers, or a pharmaceutically-acceptable addition salt thereof, or an N-oxide thereof, for the manufacture of a pharmaceutical composition for the treatment, prevention or alleviation of a disease or a disorder or a condition of a living animal body, including a human, which disorder, disease or condition is responsive to activation of KJ channels.

In another embodiment the invention provides a method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of a compound of the invention, a stereoisomer or a mixture of its stereoisomers, or a pharmaceutically-acceptable addition salt thereof, or an N-oxide thereof or a pharmaceutically-acceptable addition salt thereof.

Another embodiment of the invention is the provision of compounds with optimal pharmacodynamic and/or pharmacokinetic properties such as kinetic behaviour, bioavailability, solubility, efficacy and/or adverse effects.

Other embodiments of the invention will be apparent to the person skilled in the art from the following detailed description and examples.

### DETAILED DISCLOSURE OF THE INVENTION

In one embodiment the present invention provides compounds of Formula (I)

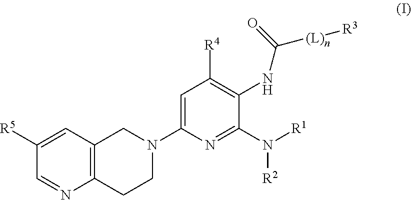

a stereoisomer or a mixture of its stereoisomers, or a pharmaceutically-acceptable addition salt thereof, or an N-oxide thereof, wherein
R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁₋₆-alkyl;
L represents a linker selected from —CR′R″—, —CH₂—CR′R″—, —CR′R″—CH₂—, and —O—, wherein R′ and R″, independently of each other, represent hydrogen, C₁₋₆-alkyl or halo;
n is 0 or 1;
R³ represents C₁₋₆-alkyl, phenyl, pyridyl, furanyl, imidazolyl, oxazolyl, isoxazolyl, thiazolyl, isothiazolyl, or C₃₋₆-cycloalkyl, which phenyl, pyridyl, furanyl, imidazolyl, oxazolyl, isoxazolyl, thiazolyl and isothiazolyl is optionally substituted one or more times with substituents selected from C₁₋₆-alkyl, C₃₋₆-cycloalkyl, phenyl, C₁₋₆-alkoxy, halo and trifluoromethyl;
R⁴ represents hydrogen, halo or C₁₋₆-alkyl; and
R⁵ represents hydrogen or halo.

In another embodiment of the invention, in formula (I), R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁₋₆-alkyl;

L represents a linker selected from —CR′R″—, —CH₂—CR′R″- and —CR′R″-CH₂—, wherein R′ and R″, independently of each other, represent hydrogen, C₁₋₆-alkyl or halo;
n is 0 or 1;
R³ represents C₁₋₆-alkyl, phenyl or furanyl, which phenyl and furanyl is optionally substituted one or more times with substituents selected from C₁₋₆-alkyl, C₁₋₆-alkoxy, halo and trifluoromethyl; and
R⁴ represents hydrogen, halo or C₁₋₆-alkyl.

In another embodiment of the invention, in formula (I), R¹ and R², together with the nitrogen to which they are attached, represent pyrrolidinyl, which is optionally substituted one or more times with a substituent selected from the group consisting of halo, hydroxy, amino, C₁₋₆-alkyl, trifluoromethyl, C₁₋₆-alkoxy, hydroxyC₁₋₆-alkyl and C₁₋₆-alkoxy-C₁₋₆-alkyl. In another embodiment R¹ and R², together with the nitrogen to which they are attached, represent pyrrolidinyl. In another embodiment R¹ and R², together with the nitrogen to which they are attached, represent pyrrolidinyl, which is substituted one or two times with a substituent selected from the group consisting of halo, hydroxy, amino, C₁₋₆-alkyl and trifluoromethyl. In another embodiment R¹ and R², together with the nitrogen to which they are attached, represent pyrrolidinyl, substituted one time with a substituent selected from the group consisting of halo, hydroxy, amino, C₁₋₆-alkyl and trifluoromethyl. In another embodiment R¹ and R², together with the nitrogen to which they are attached, represent pyrrolidinyl substituted one time with halo. In another embodiment R¹ and R², together with the nitrogen to which they are attached, represent pyrrolidinyl substituted one time with C₁₋₆-alkyl, such as methyl. In another embodiment R¹ and R², together with the nitrogen to which they are attached, represent pyrrolidinyl substituted one time with trifluoromethyl. In another embodiment R¹ and R², together with the nitrogen to which they are attached, represent pyrrolidinyl substituted two times with a substituent selected from the group consisting of halo, hydroxy, amino, C₁₋₆-alkyl and trifluoromethyl. In another embodiment R¹ and R², together with the nitrogen to which they are attached, represent pyrrolidinyl substituted two times with halo. In another embodiment R¹ and R², together with the nitrogen to which they are attached, represent pyrrolidinyl substituted two times with C₁₋₆-alkyl, such as methyl.

In another embodiment of the invention, in formula (I), R¹ and R², together with the nitrogen to which they are attached, represent piperidinyl, which is optionally substituted one or more times with a substituent selected from the group consisting of halo, hydroxy, amino, C₁₋₆-alkyl, trifluoromethyl, C₁₋₆-alkoxy, hydroxy-C₁-C₆-alkyl and C₁₋₆-alkoxy-C₁₋₆-alkyl. In another embodiment R¹ and R², together with the nitrogen to which they are attached, represent piperidinyl. In another embodiment R¹ and R², together with the nitrogen to which they are attached, represent piperidinyl, which is substituted one or two times with a substituent selected from the group consisting of halo, hydroxy, amino, C₁₋₆-alkyl and trifluoromethyl. In another embodiment R¹ and R², together with the nitrogen to which they are attached, represent piperidinyl substituted two times with a substituent selected from the group consisting of halo, hydroxy, amino, C₁₋₆-alkyl and trifluoromethyl. In another embodiment R¹ and R², together with the nitrogen to which they are attached, represent piperidinyl substituted two times with halo, such as fluoro. In another embodiment R¹ and R², together with the nitrogen to which they are attached, represent pyrrolidinyl substituted two times with C₁₋₆-alkyl, such as methyl.

In another embodiment of the invention, in formula (I), R¹ and R², together with the nitrogen to which they are attached, represent 2,5-dihydro-1H-pyrrol-1-yl.

In another embodiment of the invention, in formula (I), R¹ and R², together with the nitrogen to which they are attached, represent thiazolidinyl.

In another embodiment of the invention, in formula (I), R¹ and R², together with the nitrogen to which they are attached, represent piperidinyl, which is optionally substituted one or more times with a substituent selected from the group consisting of halo, hydroxy, amino, C₁₋₆-alkyl, trifluoromethyl, C₁₋₆-alkoxy, hydroxy-C₁-C₆-alkyl and C₁₋₆-alkoxy-C₁₋₆-alkyl.

In another embodiment of the invention, in formula (I), R¹ and R², together with the nitrogen to which they are attached, represent piperazinyl which is optionally substituted one or more times with a substituent selected from the group consisting of halo, hydroxy, amino, C₁₋₆-alkyl, trifluoromethyl, C₁₋₆-alkoxy, hydroxy-C₁-C₆-alkyl and C₁₋₆-alkoxy-C₁₋₆-alkyl.

In another embodiment of the invention, in formula (I), R¹ and R², together with the nitrogen to which they are attached, represent morpholinyl, which is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁₋₆-alkyl. In another embodiment R¹ and R², together with the nitrogen to which they are attached, represent morpholinyl. In another embodiment R¹ and R², together with the nitrogen to which they are attached, represent morpholinyl substituted one or two times with a substituent selected from the group consisting of halo, hydroxy, amino, C₁₋₆-alkyl, trifluoromethyl and C₁₋₆-alkoxy.

In another embodiment of the invention, in formula (I), R³ represents C₁₋₆-alkyl, such as methyl, ethyl, propyl or tert-butyl. In another embodiment, R³ represents tert-butyl.

In another embodiment of the invention, in formula (I), R³ represents phenyl, which is optionally substituted one or more times with substituents selected from C₁₋₆-alkyl, C₁₋₆-alkoxy halo and trifluoromethyl.

In another embodiment of the invention, in formula (I), R³ represents phenyl.

In another embodiment of the invention, in formula (I), R³ represents phenyl, which is substituted one time with a substituent selected from C₁₋₆-alkyl, C₁₋₆-alkoxy, halo and trifluoromethyl. In another embodiment R³ represents phenyl which is substituted one time with C₁₋₆-alkyl, such as methyl. In another embodiment R³ represents phenyl which is substituted one time with halo. In another embodiment R³ represents phenyl which is substituted one time with C₁₋₆-alkoxy. In another embodiment R³ represents phenyl which is substituted one time with trifluoromethyl.

In another embodiment of the invention, in formula (I), R³ represents phenyl, which is substituted two times with substituents selected from C₁₋₆-alkyl, C₁₋₆-alkoxy, halo and trifluoromethyl. In another embodiment R³ represents phenyl which is substituted two times with C₁₋₆-alkyl. In another embodiment R³ represents phenyl which is substituted two times with C₁₋₆-alkoxy. In another embodiment R³ represents phenyl which is substituted two times with halo. In another embodiment R³ represents phenyl which is substituted two times with fluoro. In another embodiment R³ represents phenyl which is substituted two times with trifluoromethyl.

In another embodiment of the invention, in formula (I), R³ represents pyridyl which is optionally substituted one or more times with substituents selected from C₁₋₆-alkyl, C₁₋₆-alkoxy halo and trifluoromethyl. In another embodiment R³ represents pyridyl substituted one or more times with C₁₋₆-alkyl.

In another embodiment of the invention, in formula (I), R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or more times with substituents selected from C₁₋₆-alkyl, C₃₋₆-cycloalkyl, phenyl, C₁₋₆-alkoxy, halo and trifluoromethyl.

In another embodiment of the invention, in formula (I), R³ represents furanyl. In another embodiment R³ represents imidazolyl. In another embodiment R³ represents oxazolyl or isoxazolyl. In another embodiment R³ represents thiazolyl or isothiazolyl.

In another embodiment of the invention, in formula (I), R³ represents furanyl, imidazolyl, oxazolyl, isoxazolyl, thiazolyl or isothiazolyl, all of which is substituted one time with a substituent selected from C₁₋₆-alkyl, C₁₋₆-alkoxy, halo and trifluoromethyl. In another embodiment R³ represents furanyl, imidazolyl, oxazolyl, isoxazolyl, thiazolyl or isothiazolyl, all of which is substituted one time with C₁₋₆-alkyl, such as methyl. In another embodiment R³ represents furanyl which is substituted one time with halo. In another embodiment R³ represents furanyl, imidazolyl, oxazolyl, isoxazolyl, thiazolyl or isothiazolyl which is substituted one time with C₁₋₆-alkoxy. In another embodiment R³ represents furanyl which is substituted one time with trifluoromethyl.

In another embodiment of the invention, in formula (I), R³ represents furanyl, imidazolyl, oxazolyl, isoxazolyl, thiazolyl or isothiazolyl, all of which is substituted two times with substituents selected from C₁₋₆-alkyl, C₁₋₆-alkoxy, halo and trifluoromethyl. In another embodiment R³ represents furanyl, imidazolyl, oxazolyl, isoxazolyl, thiazolyl or isothiazolyl, all of which is substituted two times with C₁₋₆-alkyl. In another embodiment R³ represents furanyl which is substituted two times with C₁₋₆-alkoxy. In another embodiment R³ represents furanyl which is substituted two times with halo. In another embodiment R³ represents furanyl which is substituted two times with fluoro. In another embodiment R³ represents furanyl which is substituted two times with trifluoromethyl.

In another embodiment of the invention, in formula (I), R³ represents oxazolyl or isoxazolyl, which oxazolyl or isoxazolyl is substituted one or two times with C₁₋₆-alkyl. In another embodiment R³ represents oxazolyl or isoxazolyl, which oxazolyl or isoxazolyl is substituted one time with phenyl or C₃₋₆-cycloalkyl.

In another embodiment of the invention, in formula (I), R³ represents C₃₋₆-cycloalkyl.

In another embodiment of the invention, in formula (I), L represents a linker selected from —CR′R″- and —CH₂—CR′R″—, wherein R′ and R″, independently of each other, represent hydrogen or C₁₋₆-alkyl.

In another embodiment of the invention, in formula (I), L represents a linker selected from —CH₂—, —CH₂—CH₂—, —CH(CH₃)—, —CH₂—CH(CH₃)— and —CH₂—C(CH₃)₂—. In another embodiment, L represents —CH₂—. In another embodiment, L represents —CH₂—CH₂—. In another embodiment L represents —CH(CH₃)—. In another embodiment, L represents —CH₂—CH(CH₃)—. In another embodiment L represents —CH₂—C(CH₃)₂—.

In another embodiment of the invention, in formula (I), L represents —O—.

In another embodiment of the invention, in formula (I), n is 1.

In another embodiment of the invention, in formula (I), n is 0.

In another embodiment of the invention, in formula (I), R⁴ represents hydrogen.

In another embodiment of the invention, in formula (I), R⁴ represents halo, such as chlorine.

In another embodiment of the invention, in formula (I), R⁴ represents C₁-C₆-alkyl, such as methyl.

In another embodiment of the invention, in formula (I), R⁵ represents hydrogen.

In another embodiment of the invention, in formula (I), R⁵ represents halo, such as chlorine.

In another embodiment of the invention, in formula (I), R¹ and R², together with the nitrogen to which they are attached, is pyrrolidinyl, which pyrrolidinyl is optionally substituted one or more times with a substituent selected from the group consisting of halo, C₁₋₆-alkyl and trifluoromethyl; R³ represents C₁₋₆-alkyl, phenyl or furanyl, which phenyl and furanyl is optionally substituted one or more times with substituents selected from C₁₋₆-alkyl, C₁₋₆-alkoxy, halo and trifluoromethyl; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is pyrrolidinyl, which pyrrolidinyl is optionally substituted one or more times with a substituent selected from the group consisting of halo, C₁₋₆-alkyl and trifluoromethyl; R³ represents C₁₋₆-alkyl, phenyl or furanyl, which phenyl and furanyl is optionally substituted one or more times with substituents selected from C₁₋₆-alkyl, C₁₋₆-alkoxy, halo and trifluoromethyl; n is 0; R⁴ represents C₁₋₆-alkyl and R⁵ represents hydrogen.

In another embodiment of the invention, in formula (I), R¹ and R², together with the nitrogen to which they are attached, is pyrrolidinyl, which pyrrolidinyl is substituted one time with halo; R³ represents phenyl or furanyl, which phenyl and furanyl is optionally substituted one or more times with substituents selected from C₁₋₆-alkyl and halo; L represents —CH₂—; n is 0 or 1; and R⁴ represents C₁₋₆-alkyl.

In another embodiment of the invention, in formula (I), R¹ and R², together with the nitrogen to which they are attached, is pyrrolidinyl; R³ represents C₁₋₆-alkyl or phenyl, which phenyl is substituted two times with halo; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents C₁₋₆-alkyl or phenyl, which phenyl is substituted two times with halo; L represents —CH₂—; n is 0; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents C₁-C₆-alkyl; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents C₁-C₆-alkyl; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is pyrrolidinyl or piperidinyl, which piperidinyl is substituted two times with halo; R³ represents phenyl, which phenyl is substituted one or two times with halo; L represents —CH₂—; n is 1; R⁴ represents O₁₋₆-alkyl and R⁵ represents hydrogen.

In another embodiment of the invention, in formula (I), R¹ and R², together with the nitrogen to which they are attached, is pyrrolidinyl; R³ represents phenyl, which phenyl is substituted one or two times with halo; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents phenyl, which phenyl is substituted one or two times with halo; L represents —CH₂—; n is 0; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents phenyl, which phenyl is substituted one time with halo; L represents —CH₂—; n is 0; R⁴ represents C₁₋₆-alkyl and R⁵ represents hydrogen.

In another embodiment of the invention, in formula (I), R¹ and R², together with the nitrogen to which they are attached, is pyrrolidinyl; R³ represents phenyl, which phenyl is substituted two times with halo; L represents —CH₂—; n is 1; R⁴ represents C₁₋₆-alkyl and R⁵ represents hydrogen.

In another embodiment of the invention, in formula (I), R¹ and R², together with the nitrogen to which they are attached, is piperidinyl substituted two times with halo; R³ represents pyridyl, which is substituted one time with C₁₋₆-alkyl; n is 0; R⁴ represents C₁₋₆-alkyl and R⁵ represents hydrogen.

In another embodiment of the invention, in formula (I), R¹ and R², together with the nitrogen to which they are attached, is piperidinyl substituted two times with halo; R³ represents pyridyl, which is substituted one time with C₁₋₆-alkyl; L is —O—, n is 1; R⁴ represents C₁₋₆-alkyl and R⁵ represents hydrogen.

In another embodiment of the invention, in formula (I), R¹ and R², together with the nitrogen to which they are attached, is piperidinyl substituted two times with halo, R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or two times with substituents selected from C₁₋₆-alkyl, C₃₋₆-cycloalkyl and phenyl, n is 0; R⁴ represents C₁₋₆-alkyl and R⁵ represents hydrogen.

In another embodiment of the invention, in formula (I), R¹ and R², together with the nitrogen to which they are attached, is piperidinyl substituted two times with fluoro, R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or two times with C₁₋₆-alkyl, n is 0; R⁴ represents C₁₋₆-alkyl and R⁵ represents hydrogen.

In another embodiment of the invention, in formula (I), R¹ and R², together with the nitrogen to which they are attached, is pyrrolidinyl, which pyrrolidinyl is optionally substituted one or more times with a substituent selected from the group consisting of halo and C₁₋₆-alkyl and trifluoromethyl; R³ represents C₁₋₆-alkyl, phenyl or furanyl, which phenyl and furanyl is optionally substituted one or more times with substituents selected from C₁₋₆-alkyl, C₁₋₆-alkoxy, halo and trifluoromethyl; L represents —CH₂—; n is 0 or 1; R⁴ represents C₁₋₆-alkyl and R⁵ represents halo.

In another embodiment of the invention, in formula (I), R¹ and R², together with the nitrogen to which they are attached, is pyrrolidinyl, which pyrrolidinyl is optionally substituted one or more times with a substituent selected from the group consisting of halo and C₁₋₆-alkyl and trifluoromethyl; R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or two times with substituents selected from C₁₋₆-alkyl, C₃₋₆-cycloalkyl and phenyl; L represents —CH₂—; n is 0 or 1; R⁴ represents C₁₋₆-alkyl and R⁵ represents halo.

In another embodiment of the invention, in formula (I), R¹ and R², together with the nitrogen to which they are attached, is piperidinyl substituted two times with halo; R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or two times with substituents selected from C₁₋₆-alkyl, C₃₋₆-cycloalkyl and phenyl; L represents —CH₂—; n is 0 or 1; R⁴ represents C₁₋₆-alkyl and R⁵ represents halo.

In another embodiment of the invention, in formula (I), R¹ and R², together with the nitrogen to which they are attached, is piperidinyl substituted two times with halo, R³ represents furanyl, imidazolyl, oxazolyl, isoxazolyl, thiazolyl or isothiazolyl, which furanyl, imidazolyl, oxazolyl, isoxazolyl, thiazolyl and isothiazolyl is optionally substituted one or two times with substituents selected from C₁₋₆-alkyl, C₃₋₆-cycloalkyl and phenyl, n is 0; R⁴ represents C₁₋₆-alkyl and R⁵ represents halo.

In another embodiment of the invention, in formula (I), R¹ and R², together with the nitrogen to which they are attached, is pyrrolidinyl, optionally substituted one time with halo; R³ represents C₁₋₆-alkyl; L represents —CH₂—; n is 1; R⁴ represents C₁₋₆-alkyl and R⁵ represents halo.

In another embodiment of the invention the compound of the invention is:
2-(3,5-Difluoro-phenyl)-N-[6-(7,8-dihydro-5H-[1,6]naphthyridin-6-yl)-4-methyl-2-pyrrolidin-1-yl-pyridin-3-yl]-acetamide;
N-[6-(7,8-Dihydro-5H-[1,6]naphthyridin-6-yl)-4-methyl-2-pyrrolidin-1-yl-pyridin-3-yl]-3,3-dimethyl-butyramide;
N-[6-(7,8-Dihydro-5H-[1,6]naphthyridin-6-yl)-4-methyl-2-pyrrolidin-1-yl-pyridin-3-yl]-3-fluoro-benzamide;
   or a stereoisomer or a mixture of its stereoisomers, or a pharmaceutically-acceptable addition salt thereof, or an N-oxide thereof.

In another embodiment of the invention the compound of the invention is:
N-[6-(7,8-dihydro-5H-1,6-naphthyridin-6-yl)-2-[(3R)-3-fluoropyrrolidin-1-yl]-4-methyl-3-pyridyl]-3-methyl-furan-2-carboxamide;
2-(3,5-difluorophenyl)-N-[6-(7,8-dihydro-5H-1,6-naphthyridin-6-yl)-2-[(3R)-3-fluoropyrrolidin-1-yl]-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-3-methyl-furan-2-carboxamide;
2-(3,5-difluorophenyl)-N-[2-(4,4-difluoro-1-piperidyl)-6-(7,8-dihydro-5H-1,6-naphthyridin-6-yl)-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-1-methyl-imidazole-2-carboxamide;
N-[2-(4,4-difluoro-1-piperidyl)-6-(7,8-dihydro-5H-1,6-naphthyridin-6-yl)-4-methyl-3-pyridyl]-5-methyl-oxazole-4-carboxamide;
N-[2-(4,4-difluoro-1-piperidyl)-6-(7,8-dihydro-5H-1,6-naphthyridin-6-yl)-4-methyl-3-pyridyl]-4-methyl-thiazole-5-carboxamide;
N-[2-(4,4-difluoro-1-piperidyl)-6-(7,8-dihydro-5H-1,6-naphthyridin-6-yl)-4-methyl-3-pyridyl]-3-methyl-isoxazole-4-carboxamide;
N-[2-(4,4-difluoro-1-piperidyl)-6-(7,8-dihydro-5H-1,6-naphthyridin-6-yl)-4-methyl-3-pyridyl]-4-methyl-oxazole-5-carboxamide;
2-cyclopropyl-N-[2-(4,4-difluoro-1-piperidyl)-6-(7,8-dihydro-5H-1,6-naphthyridin-6-yl)-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-5-ethyl-oxazole-4-carboxamide;
N-[2-(4,4-difluoro-1-piperidyl)-6-(7,8-dihydro-5H-1,6-naphthyridin-6-yl)-4-methyl-3-pyridyl]-3-methyl-pyridine-2-carboxamide;
N-[2-(4,4-difluoro-1-piperidyl)-6-(7,8-dihydro-5H-1,6-naphthyridin-6-yl)-4-methyl-3-pyridyl]-2,5-dimethyl-oxazole-4-carboxamide;
N-[2-(4,4-difluoro-1-piperidyl)-6-(7,8-dihydro-5H-1,6-naphthyridin-6-yl)-4-methyl-3-pyridyl]-5-phenykoxazole-4-carboxamide;
5-cyclopropyl-N-[2-(4,4-difluoro-1-piperidyl)-6-(7,8-dihydro-5H-1,6-naphthyridin-6-yl)-4-methyl-3-pyridyl]oxazole-4-carboxamide;
N-[2-(4,4-difluoro-1-piperidyl)-6-(7,8-dihydro-5H-1,6-naphthyridin-6-yl)-4-methyl-3-pyridyl]-5-methyl-isothiazole-4-carboxamide;
ethyl N-[2-(4,4-difluoro-1-piperidyl)-6-(7,8-dihydro-5H-1,6-naphthyridin-6-yl)-4-methyl-3-pyridyl]carbamate;
   or a stereoisomer or a mixture of its stereoisomers, or a pharmaceutically-acceptable addition salt thereof, or an N-oxide thereof.

In another embodiment of the invention the compound of the invention is:
N-[6-(3-chloro-7,8-dihydro-5H-1,6-naphthyridin-6-yl)-2-[(3R)-3-fluoropyrrolidin-1-yl]-4,6-dimethyl-1H-pyridin-3-yl]-3,3-dimethyl-butanamide; or a stereoisomer or a mixture of its stereoisomers, or a pharmaceutically-acceptable addition salt thereof, or an N-oxide thereof.

Any combination of two or more of the embodiments described herein is considered within the scope of the present invention.

### Definition of Terms

As used throughout the present specification and appended claims, the following terms have the indicated meaning:

The term “C₁₋₆-alkyl” as used herein means a saturated, branched or straight hydrocarbon group having from 1-6 carbon atoms, e.g. C₁₋₃-alkyl, C₁₋₄-alkyl, C₁₋₆-alkyl, C₂₋₆-alkyl, C₃₋₆-alkyl, and the like. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), and the like.

The term “halo” or “halogen” means fluorine, chlorine, bromine or iodine.

The term “hydroxy” shall mean the radical —OH.

The term “amino” shall mean the radical —NH₂.

The term “trihalomethyl” means trifluoromethyl, trichloromethyl, and similar trihalo-substituted methyl groups.

The term “C₁₋₆-alkoxy” as used herein refers to the radical —O—C₁₋₆-alkyl. Representative examples are methoxy, ethoxy, propoxy (e.g. 1-propoxy, 2-propoxy), butoxy (e.g. 1-butoxy, 2-butoxy, 2-methyl-2-propoxy), pentoxy (1-pentoxy, 2-pentoxy), hexoxy (1-hexoxy, 3-hexoxy), and the like.

The term “hydroxy-C₁₋₆-alkyl” as used herein refers to alkyl substituted one or more times at any carbon atom(s) with hydroxyl. Representative examples are hydroxymethyl, hydroxyethyl (e.g. 1-hydroxyethyl, 2-hydroxyethyl) and the like.

The term “C₁₋₆-alkoxy-C₁₋₆-alkyl” as used herein refers to an C₁₋₆-alkyl-O—C₁₋₆-alkyl group, wherein the C₁₋₆-alkyl and C₁₋₆-alkyl-O— are as defined above. Representative examples are methoxy-methyl, methoxy-ethyl, ethoxy-methyl, and ethoxy-ethyl.

The term “C₃₋₆-cycloalkyl” as used herein refers to represents a saturated monocyclic carbocyclic ring having from 3 to 6 carbon atoms, e.g. C₃₋₄-alkyl, C₃₋₅-alkyl, and the like. Representative examples are cyclopropyl, cyclobutyl, cyclopentyl, cyclohexyl, and the like.

The term “optionally substituted” as used herein means that the groups in question are either unsubstituted or substituted with one or more of the substituents specified. When the group(s) in question is/are substituted with more than one substituent the substituents may be the same or different.

Certain of the defined terms may occur more than once in the structural formulae, and upon such occurrence each term shall be defined independently of the other.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The terms “disease”, “condition” and “disorder” as used herein are used interchangeably to specify a state of a patient which is not the normal physiological state of man.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

The term “effective amount” as used herein means a dosage which is sufficient in order for the treatment of the patient to be effective compared with no treatment.

The term “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

### Pharmaceutically Acceptable Salts

The compounds of the invention may be provided in any form suitable for the intended administration. Suitable forms include pharmaceutically (i.e. physiologically) acceptable salts, and pre- or prodrug forms of the compounds of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycollate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Steric Isomers

The compounds of the present invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The compounds of the present invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The compounds of the invention have been found useful as modulators of the KJ (KCNQ) potassium channels. At present five such channels are known, i.e. the Kᵥ7.1 (KCNQ1) channel, the Kᵥ7.2 (KCNQ2) channel, the Kᵥ7.3 (KCNQ3) channel, the Kᵥ7.4 (KCNQ4) channel, and the Kᵥ7.5 (KCNQ5) channel, and heteromeric combinations hereof. Moreover, the modulatory activity may be inhibitory (i.e. inhibitory activity) or stimulatory (i.e. activating activity).

The modulatory activity may be determined using conventional methods, e.g. binding or activity studies, known in the art, e.g. as described in WO 2004/080377 (NeuroSearch NS).

In one aspect of the invention, the compounds of the invention show stimulating activity at Kᵥ7.2, Kᵥ7.3, Kᵥ7.4 and/or Kᵥ7.5 potassium channels, and heteromeric combinations hereof. Compounds of the invention are selective, e.g. showing Kᵥ7.2, Kᵥ7.2+Kᵥ7.3, and/or Kᵥ7.4 potassium channel activation.

Accordingly, the compounds of the invention are considered useful for the treatment, prevention or alleviation of a disease or a disorder or a condition of a living animal body, including a human, which disorder, disease or condition is responsive to modulation of a KJ potassium channel.

Due to the distribution of KCNQ channels within the organism, KCNQ channel modulators are considered useful for the treatment or alleviation of conditions as diverse as an affective disorder, a neuro-physiological disorder, an anxiety disorder, depression, a bipolar disorder, a sleep disorder, addiction, an eating disorder, a phobia, a neurodegenerative disorder, Parkinson's disease, a mood disorder, a psychotic disorder, a compulsive behaviour, mania, psychosis, schizophrenic, dementia, Alzheimer's disease, epilepsy, convulsions, seizure disorders, absence seizures, vascular spasms, coronary artery spasms, tremor, muscle spasms, myasthenia gravis, a motor neuron disease, motion and motor disorders, a tic disorder, a Parkinson-like motor disorder, essential tremors, multiple sclerosis, amyelotrophic lateral sclerosis (ALS), multiple system atrophy, corticobasal degeneration, HIV associated dementia, Huntington's disease, Pick's disease, torsades de pointes, functional bowel disorders, CNS damage caused by trauma, stroke or neurodegenerative illness or diseases, ataxia, myokymia, spasticity, myopathy, learning and cognitive disorders, memory dysfunction, memory impairment, age-associated memory loss, Down's syndrome, pain, acute or chronic pain, mild pain, moderate or severe pain, neuropathic pain, central pain, pain related to diabetic neuropathy, to postherpetic neuralgia, to peripheral nerve injury, somatic pain, visceral pain or cutaneous pain, pain caused by inflammation or by infection, postoperative pain, phantom limb pain, neuronal hyperexcitability disorders, peripheral nerve hyperexcitability, chronic headache, migraine, migraine-related disorders, tension-type headache, heart failure, cardiac disorders, cardiomyopathia, cardiac arrhythmia, cardiac ischaemia, long QT syndrome, inflammatory diseases or conditions, inflammatory bowel disease, Crohn's disease, ulcerative colitis, Creutzfeld-Jacobs disease, an obstructive or inflammatory airway disease, asthma, an airway hyper reactivity, pneumoconiosis, aluminosis, anthracosis, asbestosis, chalicosis, ptilosis, siderosis, silicosis, tabacosis, byssinosis, chronic obstructive pulmonary disease (COPD), excerbation of airways hyper reactivity, cystic fibrosis, hearing impairment or hearing loss, progressive hearing loss, tinnitus, a drug-dependence or drug-addiction disorder, hyperactive gastric motility, ophthalmic conditions, erectile dysfunction, fibromylgia, for inducing or maintaining bladder control, nocturia, bladder spasms, overactive bladder (OAB), bladder outflow obstruction, interstitial cystitis (IC) (also called painfull bladder syndrome) and urinary incontinence.

In another embodiment the disease, disorder or condition contemplated according to the invention is an anxiety disorder such as panic disorder, agoraphobia, phobias, social anxiety disorder, obsessive-compulsive disorder and posttraumatic stress disorder. In another embodiment the disease, disorder or condition contemplated according to the invention is anxiety. In another embodiment the disease, disorder or condition contemplated according to the invention is schizophrenia.

In one embodiment the compounds of the invention are considered useful for treatment, prevention or alleviation of a disease, disorder or adverse condition of the CNS. In another embodiment, the disease, disorder or condition is an affective disorder, a neuro-physiological disorder, an anxiety disorder, depression, a bipolar disorder, a sleep disorder, addiction, an eating disorder, a phobia, a neurodegenerative disorder, Parkinson's disease, a mood disorder, a psychotic disorder, a compulsive behaviour, mania, psychosis or schizophrenia. In another embodiment the compounds of the invention are useful for the treatment or alleviation of schizophrenia. In another embodiment the compounds of the invention are useful for the treatment or alleviation of depression. In another embodiment the compounds of the invention are useful for the treatment or alleviation of bipolar disorder.

In another embodiment the compounds of the invention are considered useful for treatment, prevention or alleviation of a CNS damage caused by trauma or by a spinal cord damage, stroke, traumatic brain injury, a neurodegenerative illness or disease, dementia, Alzheimer's disease, a motor neuron disease, a Parkinson-like motor disorder, multiple sclerosis, amyelotrophic lateral sclerosis (ALS), multiple system atrophy, HIV associated dementia, Huntington's disease, Pick's disease, torsades de pointes, tremor, muscle spasms, myasthenia gravis, convulsions, ataxia, myokymia, seizures, epilepsy or spasticity. In another embodiment the compounds of the invention are useful for the treatment or alleviation of epilepsy.

In another embodiment the compounds of the invention are considered useful for treatment, prevention or alleviation of pain, including acute and chronic pain, mild pain, moderate or even severe pain of acute, chronic or recurrent character, as well as postoperative pain, phantom limb pain, chronic headache, post therapeutic neuralgia, neuropathic pain, central pain, or pain related to diabetic neuropathy, to postherpetic neuralgia, to peripheral nerve injury or drug addiction, migraine and migraine-related disorders and to tension-type headache. In another embodiment the pain is somatic pain, incl. visceral pain or cutaneous pain, or pain caused by inflammation or by infection. In another embodiment the pain is neuropathic, e.g. caused by injury to the central or peripheral nervous system, e.g. due to tissue trauma, infection, diabetes, an autoimmune disease, arthritis or neuralgia.

In another embodiment the compounds of the invention are useful for the treatment or alleviation of pain. In another embodiment the compounds of the invention are useful for the treatment or alleviation of neuropathic pain.

In another embodiment the compounds of the invention are considered useful for treatment, prevention or alleviation of addiction, e.g. drug addiction, drug abuse, cocaine abuse, nicotine abuse, tobacco abuse, alcohol addiction or alcoholism, or withdrawal symptoms caused by the termination of abuse of chemical substances, in particular opioids, heroin, cocaine and morphine, benzodiazepines and benzodiazepine-like drugs, and alcohol.

In another embodiment the compounds of the invention are considered useful for treatment, prevention or alleviation of a learning and cognitive disorder, memory dysfunction, memory impairment, age-associated memory loss or Down's syndrome. In another embodiment the compounds of the invention are useful for the treatment or alleviation of cognition.

In another embodiment the compounds of the invention are considered useful for treatment, prevention or alleviation of chronic headache, migraine, migraine-related disorders or tension-type headache. In another embodiment the compounds of the invention are considered useful for treatment or alleviation of migraine.

In another embodiment the compounds of the invention are considered useful for treatment, prevention or alleviation of a disease, disorder or condition associated with the heart or skeletal muscle, heart failure, cardiomyopathia, cardiac arrhythmia, cardiac ischaemia or long QT syndrome.

In another embodiment the compounds of the invention are considered useful for treatment, prevention or alleviation of an inflammatory disease or condition, inflammatory bowel disease, Crohn's disease, ulcerative colitis or Creutzfeld-Jacobs disease.

In another embodiment the compounds of the invention are considered useful for treatment, prevention or alleviation of asthma, an obstructive or inflammatory airway disease, an airway hyper reactivity, a pneumoconiosis such as aluminosis, anthracosis, asbestosis, chalicosis, ptilosis, siderosis, silicosis, tabacosis and byssinosis, a chronic obstructive pulmonary disease (COPD), excerbation of airways hyper reactivity or cystic fibrosis. In another embodiment the compounds of the invention are considered useful for treatment or alleviation of asthma.

In another embodiment the compounds of the invention are considered useful for treatment, prevention or alleviation of progressive hearing loss or tinnitus.

In another embodiment the compounds of the invention are considered useful for treatment, prevention or alleviation of an ophthalmic disorder, a drug-dependence or drug-addiction disorder or hyperactive gastric motility.

In another embodiment the compounds of the invention are considered useful for treatment, prevention or alleviation of nocturia, bladder spasms, overactive bladder (OAB), interstitial cystitis (IC) and urinary incontinence. In another embodiment the compounds of the invention are considered useful for treatment or alleviation of urinary incontinence.

### Pharmaceutical Compositions

Viewed from one aspect the invention relates to the use of a compound of the invention, or a pharmaceutically-acceptable addition salt thereof, for the manufacture of a pharmaceutical composition for the treatment, prevention or alleviation of a disease or a disorder or a condition of a mammal, including a human, which disease, disorder or condition is responsive to modulation of Kᵥ7 channels.

Viewed from another aspect, the invention provides pharmaceutical compositions comprising a therapeutically-effective amount of a compound of the invention, or a pharmaceutically-acceptable addition salt thereof, together with at least one pharmaceutically-acceptable carrier or diluent, for the treatment, prevention or alleviation of a disease or a disorder or a condition that is responsive to modulation of Kᵥ7 channels.

While a compound for use according to the invention may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In another embodiment, the invention provides pharmaceutical compositions comprising a compound of the invention, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suite the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition may be prepared by the skilled person using standard and conventional techniques appropriate for the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additionnal active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspendsions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch compounds such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, e.g. from about 1 to about 100 mg, or from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to activation of Kᵥ7 channels, and which method comprises administering to such a living animal body, including a human, in need thereof an effective amount of a compound of the invention.

The preferred medical indications contemplated according to the invention are those stated above.

It is at present contemplated that suitable dosage ranges are 0.1 to 2000 milligrams daily, 10-1000 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 30 mg/kg i.v. and 500 mg/kg p.o. Preferred ranges are from about 0.001 to about 100 mg/kg i.v. and from about 0.1 to about 30 mg/kg p.o.

### EXAMPLES

The following examples and general procedures refer to intermediate compounds and final products for general Formula (I) identified in the specification and in the synthesis schemes. The preparation of the compounds of general Formula (I) of the present invention is described in detail using the following examples. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials.

### DCM: Dichloromethane

### DMSO: Dimethylsulfoxide

EtOAc: Ethyl acetate
Hrs: hours

### THF: Tetrahydrofuran

### TEA: Triethylamine

### MeCN: Acetonitrile

POCl₃: Phosphorus oxychloride
RaNi: Raney Nickel 50% slurry in water
Rt: Room temperature (20-25° C.)

### Preparative Example

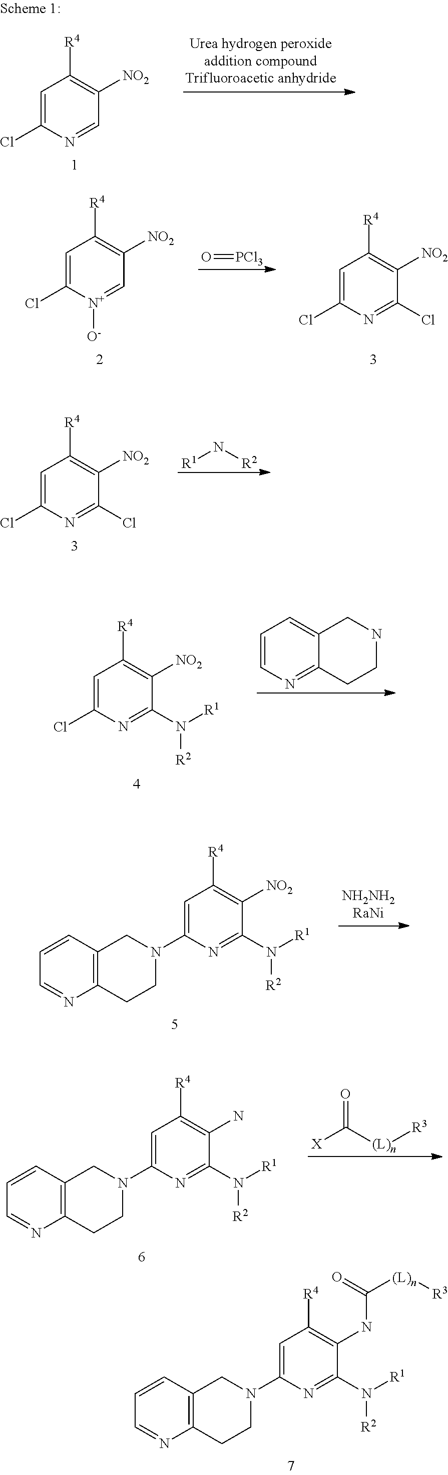

wherein R¹, R², R³, R⁴, L and n are as defined above and L is a leaving group.

### 2-Chloro-4-methyl-5-nitro-pyridine 1-oxide (Intermediate Compound)

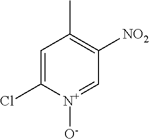

To a solution of 2-chloro-4-methyl-5-nitropyridine (10 g; 58 mmol) and urea hydrogen peroxide (ca. 12 g; 2.1 eq) in 100 mL DCM at 0° C. was added trifluoroacetic anhydride (1.6 mL; 2 eq) in a dropwise manner at a pace that allowed the reaction mixture to slowly reach room temperature. The reaction mixture was stirred for 3 h after which the bright yellow reaction mixture was added one aliquot water and stirred for another 20 min. The DCM layer was separated and the remaining aqueous layer extracted with DCM (3×150 mL). The combined organic fractions were washed with sat. NaHCO₃(aq), dried over Na₂SO₄, filtered and concentrated in vacuo yielding 9.7 g of crude material as a yellow solid. (purity-87% (GC-MS)) The solid was taken as such for the next step

### 2,6-Dichloro-4-methyl-3-nitro-pyridine (Intermediate Compound)

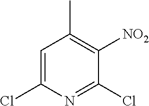

Crude 2-chloro-4-methyl-5-nitro-pyridine 1-oxide (see previous reaction; 9.7 g; 51.4 mmol) was dissolved in 50 mL POCl₃ and stirred at 80° C. overnight. The reaction mixture was poured out on 500 g ice (very exothermic!) and was stirred for 1 h in order to quench all POCl₃. The mixture was extracted with EtOAc (3×150 mL). The combined organic layers were washed with sat. NaHCO₃(aq), brine, dried over Na₂SO₄, filtered and evaporated to dryness to yield 8.73 g of crude material as a brownish liquid. (purity˜80% (GC-MS))

### 6-Chloro-4-methyl-3-nitro-2-pyrrolidin-1-yl-pyridine (Intermediate Compound)

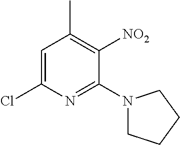

To a solution of crude 2,6-dichloro-4-methyl-3-nitro-pyridine (see previous reaction; 23.87 g; 115 mmol), pyrrolidine (11 mL; 1.1 eq) in 100 mL acetonitrile was added TEA (ca. 50 mL; 3 eq). After stirring overnight at room temperature one aliquot of water was added to the reaction mixture and the acetonitrile was removed in vacuo. The remaining aqueous layer was extracted with EtOAc (3×150 mL). The combined organic layers were dried over Na₂SO₄, filtered and concentrated in vacuo to yield a dark semi-solid. The crude material was purified by flash column chromatography (5-40% EtOAc in heptane) to yield 10 g (26% over three steps) of the title compound as a yellow solid.

### 6-Chloro-2-((R)-3-fluoro-pyrrolidin-1-yl)-4-methyl-3-nitro-pyridine (Intermediate Compound)

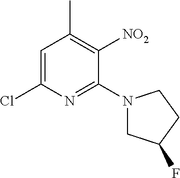

To a solution of 2,6-dichloro-4-methyl-3-nitropyridine (6.5 g; 31.4 mmol) and TEA (10 g; 3 eq) in acetonitrile (200 ml) was added (R)-(−)-3-fluoropyrrolidine hydrochloride (4.75 g; 1.2 eq). The mixture was stirred for 4 h at rt, after which the reaction mixture was quenched with sat NaHCO₃(aq) (300 ml), diluted with water and EtOAc. The layers were separeted and the waterlayer was extracted with EtOAc (3×150 mL) untill no UV (254 nm) active material was extracted). The combined organic layers were washed with brine and dried over Na₂SO₄(s). Filtration and in vacuo concentration resulted in a quantitative yield 8.36 g of the title compound as a yellow/orange oil.

### 6-chloro-2-(4,4-difluoro-1-piperidyl)-4-methyl-3-nitro-pyridine (Intermediate Compound)

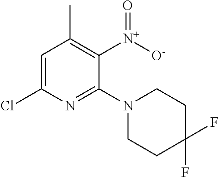

A solution of 2,6-dichloro-4-methyl-3-nitropyridine (4.5 g; 21.7 mmol), 4,4-Difluoropiperidine (3.97 g; 1.1 eq) and TEA (12 ml; ca.4 eq) in MeCN (30 ml) was stirred at rt over night after which the reaction mixture was quenched with sat.NaHCO₃(aq) solution and extracted with EtOAc (3×50 mL). The combined or ganic. were washed with brine, dried over Na₂SO₄(s), filtered and evaporated in vacuo to give 6.27 g yellow solid (90% pure title compound)

### 6-(4-Methyl-5-nitro-6-pyrrolidin-1-yl-pyridin-2-yl)-5,6,7,8-tetrahydro-[1,6]-naphthyridine (Intermediate Compound)

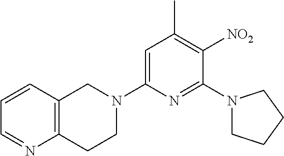

A solution of 6-chloro-4-methyl-3-nitro-2-pyrrolidin-1-yl-pyridine (1 g; 4.14 mmol) and 5,6,7,8-Tetrahydro-[1,6]naphthyridine (0.83 g; 6.2 mmol) and TEA (1.76 mL; 12.4 mmol) in DMSO (3 mL) was heated to 110° C. in a Biotage microwave for 8 hrs after which the complete reaction mixture was poured out in water and extracted with EtOAc (×3). The combined organic layers were dried over Na₂SO₄, filtered and concentrated in vacuo to yield ˜1.56 g (quant yield) of a red sticky oil.

### 6-[6-[(3R)-3-fluoropyrrolidin-1-yl]-4-methyl-5-nitro-2-pyridyl]-7,8-dihydro-5H-1,6-naphthyridine (Intermediate Compound)

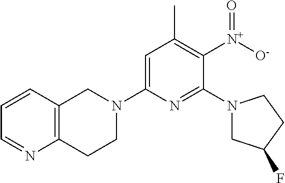

6-Chloro-2-((R)-3-fluoro-pyrrolidin-1-yl)-4-methyl-3-nitro-pyridine (0.7 g; 2.56 mmol) 5,6,7,8-tetrahydro-[1,6]naphthyridine (0.687 g; 5.1 mmol) and TEA (1.09 mL; 7.7 mmol) was added to a vial, slurrified in 5 mL DMSO, capped and heated in a microwave synthesizer (at 110° C. for 4 hrs) after which the reaction mixture was added another 2.6 mmol (1 eq; 0.36 mL) TEA and heated at 110° C. for an additional 4 hrs. The reaction mixture was poured into saturated aqueous NaHCO₃ solution (150 mL) and extracted four times with EtOAc. The combined organic phases were washed four times with brine, dried over Na₂SO₄, filtered and evaporated in vacuo. The crude material was purified by flash chromatography (50 gr silica, gradient elution: 12% EtOAc in heptane to 100%, loaded with DCM) to give 490 mg 844%) of an orange solid identical with the title compound

### 6-[6-(4,4-difluoro-1-piperidyl)-4-methyl-5-nitro-2-pyridyl]-7,8-dihydro-5H-1,6-naphthyridine (Intermediate Compound)

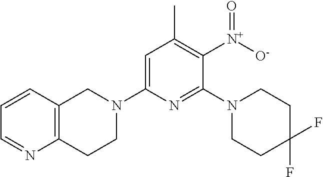

A slurry of 6-chloro-2-(4,4-difluoro-1-piperidyl)-4-methyl-3-nitro-pyridin (4.5 g; 13.88 mmol), 5,6,7,8-tetrahydro-[1,6]naphthyridine (2.24 g; 1.2 eq) and TEA (5.9 mL; 3 eq) was dissolved in DMSO (12 mL) and heated to 110° C. in a microwave synthesizer for 8 hours after which the reaction mixture was quenched with sat NaHCO₃(aq) [100 mL], diluted with water [200 mL] and 100 mL EtOAc. The layers were separated and the water layer was extracted with EtOAc (3×100 mL). The combined organic layers were washed with brine, dried over Na₂SO₄(s), filtered and concentrated in vacuo to yield a orange sticky solid. The crude material was purified by flash chromatography (silica 60A; particle size 20-40 micron; 50 g; loaded with 15% eluent and DCM) and gradient of 15-50% EtOAc in hept as the eluent resulting in 5.8 g pure title compound as an orange solid. Mp 168-169° C.

### (3,5-Difluorophenyl)-acetyl Chloride (Intermediate Compound)

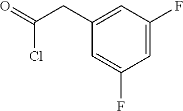

3,5-Difluorophenylacetic acid (47.5 g, 273 mmol) was dissolved in dry DCM (300 mL) followed by addition of oxalyl chloride (26.2 mL, 301 mmol). A nitrogen atmosphere was established and dry DMF (few drops) was added. The mixture was stirred overnight at RT followed by evaporation in vacuo to yield the crude title compound (52.0 g, 100%). The product was used without further purification.

### Example 1

### 2-(3,5-Difluoro-phenyl)-N-[6-(7,8-dihydro-5H-[1,6]naphthyridin-6-yl)-4-methyl-2-pyrrolidin-1-yl-pyridin-3-yl]-acetamide (Compound 1.1)

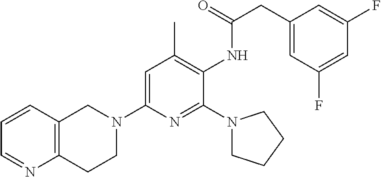

To a solution of 6-(4-methyl-5-nitro-6-pyrrolidin-1-yl-pyridin-2-yl)-5,6,7,8-tetrahydro-[1,6]naphthyridine (0.5 g; 1.47 mmol) in THF (˜25 mL) was added RaNi (Ca 2 mL) and hydrazine monohydrate (220 μl; 4.4 mmol). The slurry was stirred for 2 hrs at rt after which the reaction mixture was rapidly filtered through a plug of Hyflo into a solution of (3,5-difluorophenyl)-acetyl chloride (310 mg; 1.1 eq) in THF, which reaction mixture was stirred over the weekend. The reaction mixture was evaporated to dryness and purified by reverse phase column chromatography. (5-95% MeCN in H₂O, loaded with MeCN). The relevant fractions were collected and trituated with toluene (×3) to give 261 mg (38%) of the title product as a white solid. Mp=203-206° C. LC-ESI−HRMS of [M+H]+ shows 464.22623. Calc 464.225646.

The following compounds were synthesized employing a similar method to the one described above:

| | | | LC-ESI- | LC-ESI- |
|---|---|---|---|---|
| | | | HRMS | HRMS |
| | | | Meas. | Calc. |
| No | Structure | Name | (Da) | (Da) |
| 1.2 | 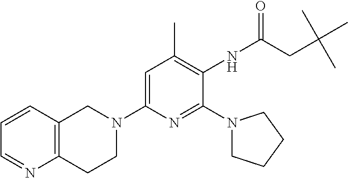 | N-[6-(7,8-Dihydro-5H- [1,6]naphthyridin-6-yl)-4- methyl-2-pyrrolidin-1-yl- pyridin-3-yl]-3,3-dimethyl- butyramide | 408.2753 | 408.2757 |
| 1.3 | 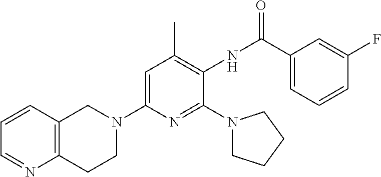 | N-[6-(7,8-Dihydro-5H- [1,6]naphthyridin-6-yl)-4- methyl-2-pyrrolidin-1-yl- pyridin-3-yl]-3-fluoro- benzamide | 432.219 | 432.2194 |
| 1.4 | 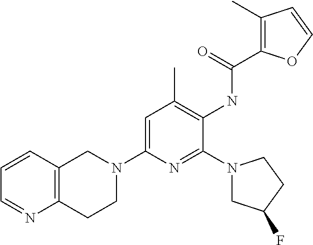 | N-[6-(7,8-dihydro-5H-1,6- naphthyridin-6-yl)-2-[(3R)- 3-fluoropyrrolidin-1-yl]-4- methyl-3-pyridyl]-3- methyl-furan-2- carboxamide | 436.2142 | 436.2143 |
| 1.5 | 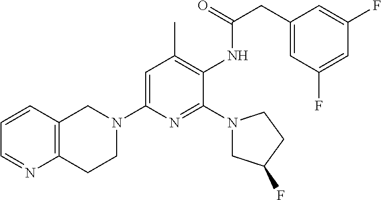 | 2-(3,5-difluorophenyl)-N- [6-(7,8-dihydro-5H-1,6- naphthyridin-6-yl)-2-[(3R)- 3-fluoropyrrolidin-1-yl]-4- methyl-3-pyridyl]- acetamide | 482.2165 | 482.2162 |
| 1.6 | 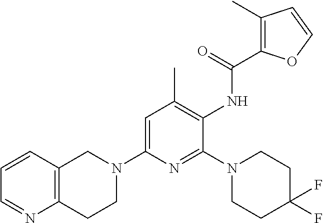 | N-[2-(4,4-difluoro-1- piperidyl)-6-(7,8-dihydro- 5H-1,6-naphthyridin-6-yl)- 4-methyl-3-pyridyl]-3- methyl-furan-2- carboxamide | 468.2204 | 468.2206 |
| 1.7 | 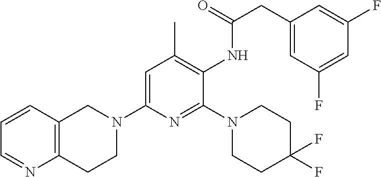 | 2-(3,5-difluorophenyl)-N- [2-(4,4-difluoro-1- piperidyl)-6-(7,8-dihydro- 5H-1,6-naphthyridin-6-yl)- 4-methyl-3-pyridyl]- acetamide | 514.2221 | 514.2224 |
| 1.8 | 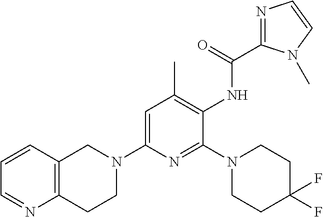 | N-[2-(4,4-difluoro-1- piperidyl)-6-(7,8-dihydro- 5H-1,6-naphthyridin-6-yl)- 4-methyl-3-pyridyl]-1- methyl-imidazole-2- carboxamide | 468.2313 | 468.2317 |
| 1.9 | 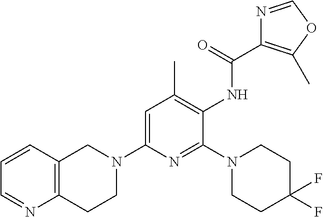 | N-[2-(4,4-difluoro-1- piperidyl)-6-(7,8-dihydro- 5H-1,6-naphthyridin-6-yl)- 4-methyl-3-pyridyl]-5- methyl-oxazole-4- carboxamide | 469.2155 | 469.2158 |
| 1.10 | 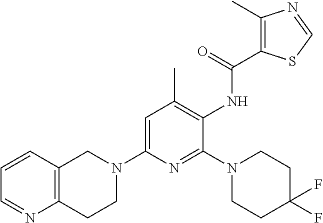 | N-[2-(4,4-difluoro-1- piperidyl)-6-(7,8-dihydro- 5H-1,6-naphthyridin-6-yl)- 4-methyl-3-pyridyl]-4- methyl-thiazole-5- carboxamide | 485.1922 | 485.1929 |
| 1.11 | 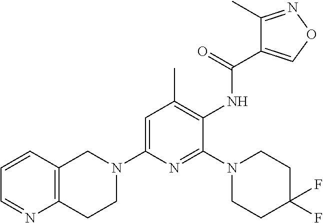 | N-[2-(4,4-difluoro-1- piperidyl)-6-(7,8-dihydro- 5H-1,6-naphthyridin-6-yl)- 4-methyl-3-pyridyl]-3- methyl-isoxazole-4- carboxamide | 469.2145 | 469.2158 |
| 1.12 | 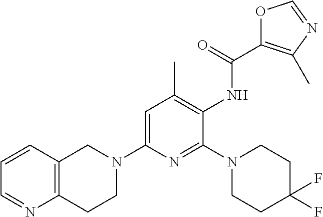 | N-[2-(4,4-difluoro-1- piperidyl)-6-(7,8-dihydro- 5H-1,6-naphthyridin-6-yl)- 4-methyl-3-pyridyl]-4- methyl-oxazole-5- carboxamide | 469.2153 | 469.2158 |
| 1.13 | 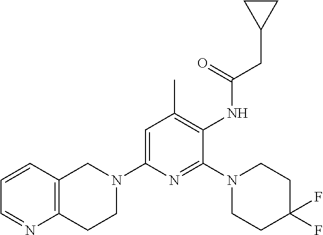 | 2-cyclopropyl-N-[2-(4,4- difluoro-1-piperidyl)-6- (7,8-dihydro-5H-1,6- naphthyridin-6-yl)-4- methyl-3-pyridyl]- acetamide | 442.2407 | 442.2412 |
| 1.14 | 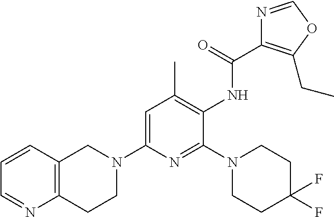 | N-[2-(4,4-difluoro-1- piperidyl)-6-(7,8-dihydro- 5H-1,6-naphthyridin-6-yl)- 4-methyl-3-pyridyl]-5- ethyl-oxazole-4- carboxamide | 483.2308 | 483.2314 |
| 1.15 | 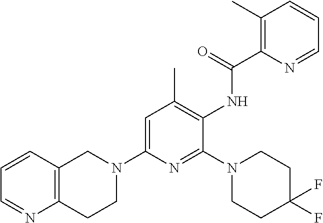 | N-[2-(4,4-difluoro-1- piperidyl)-6-(7,8-dihydro- 5H-1,6-naphthyridin-6-yl)- 4-methyl-3-pyridyl]-3- methyl-pyridine-2- carboxamide | 479.2366 | 479.2365 |
| 1.16 | 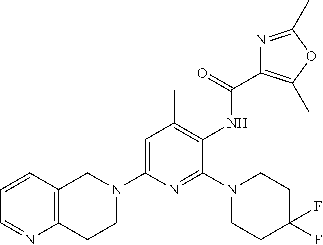 | N-[2-(4,4-difluoro-1- piperidyl)-6-(7,8-dihydro- 5H-1,6-naphthyridin-6-yl)- 4-methyl-3-pyridyl]-2,5- dimethyl-oxazole-4- carboxamide | 483.2314 | 483.2314 |
| 1.17 | 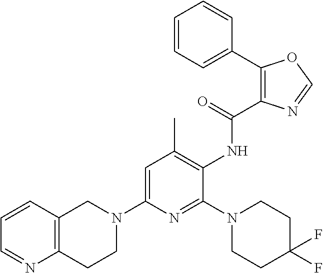 | N-[2-(4,4-difluoro-1- piperidyl)-6-(7,8-dihydro- 5H-1,6-naphthyridin-6-yl)- 4-methyl-3-pyridyl]-5- phenyl-oxazole-4- carboxamide | 531.2319 | 531.2314 |
| 1.18 | 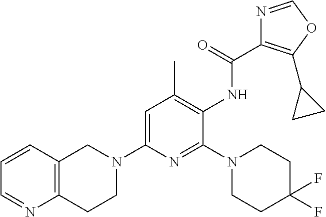 | 5-cyclopropyl-N-[2-(4,4- difluoro-1-piperidyl)-6- (7,8-dihydro-5H-1,6- naphthyridin-6-yl)-4- methyl-3-pyridyl]oxazole- 4-carboxamide | 495.2318 | 495.2314 |
| 1.19 | 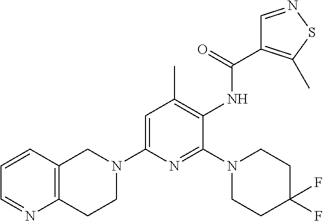 | N-[2-(4,4-difluoro-1- piperidyl)-6-(7,8-dihydro- 5H-1,6-naphthyridin-6-yl)- 4-methyl-3-pyridyl]-5- methyl-isothiazole-4- carboxamide | 485.1924 | 485.1929 |
| 1.20 | 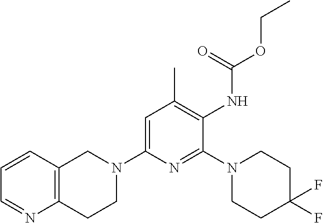 | ethyl N-[2-(4,4-difluoro-1- piperidyl)-6-(7,8-dihydro- 5H-1,6-naphthyridin-6-yl)- 4-methyl-3-pyridyl]- carbamate | 432.2211 | 432.2205 |

### Preparative Example

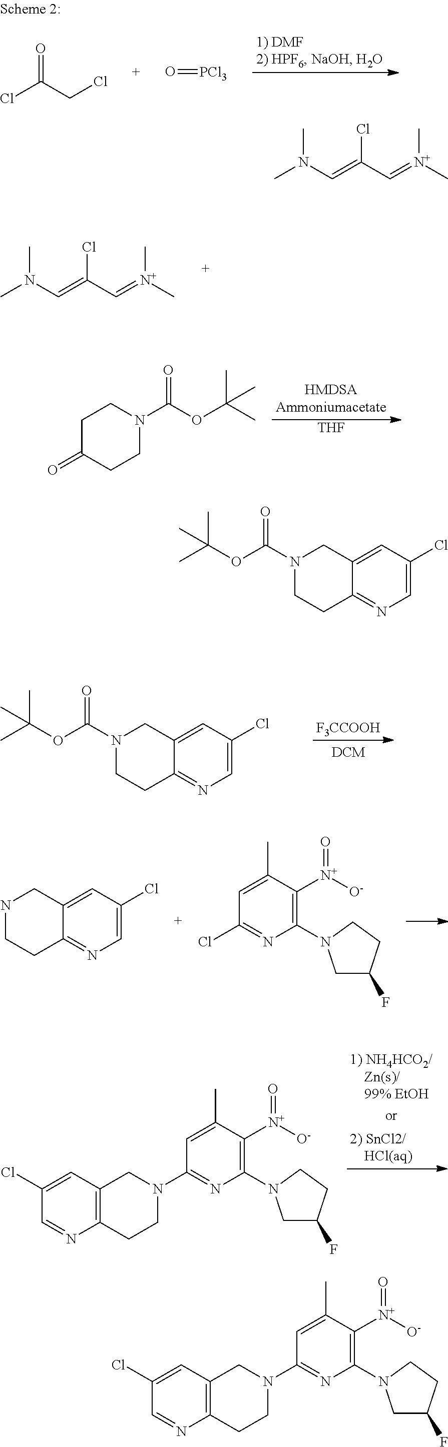

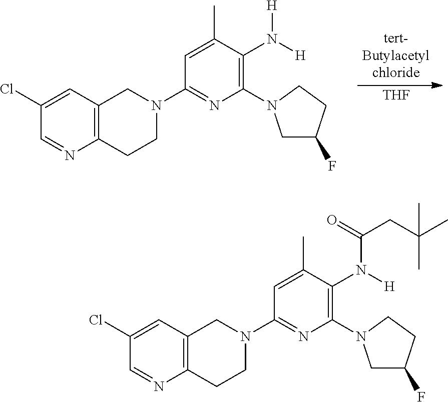

### [(Z)-2-chloro-3-dimethylamino-prop-2-enylidene]-dimethyl-ammonium (Intermediate Compound)

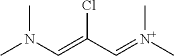

Step 1: To a cooled (0° C.). solution of chloroacetylchloride (5 g; 44.3 mmol) in DMF (21 mL) under a nitrogen atmosphere was dropwise added phosphoroxychloride (6.79 g; 44.3 mmol) over 40 min keeping the temperature at 0° C. The reaction was then heated to 70° C. for 3 hrs.
Step 2: In another reaction flask was hexafluorophosphoric acid (11.85 g; 48.70 mmol) added to ice-cold water (50 mL) and then added NaOH (aq) (5 M; 15 mL; 190 mmol). The mixture was cooled below 10° C. and then added the reaction mixture from step 1 together with NaOH (aq) (5 M; 23 mL) keeping the temp. below 10° C. Precipitation was allowed to occur over 1 h after which the title compound was isolated by filtration and washed with ice-cold water to give 11.9 g yellow solid.

### Tert-butyl 3-chloro-7,8-dihydro-5H-1,6-naphthyridine-6-carboxylate (Intermediate Compound)

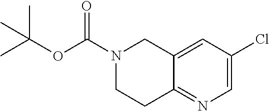

A solution of [(Z)-2-Chloro-3-dimethylamino-prop-2-enylidene]-dimethyl-ammonium (5.0 g; 16.3 mmol) in THF was cooled to −10° C. after which Boc-piperidone (2.17; 10.87 mmol) was carefully added over 25 min. The resultant solution was stirred for 1 h while the temperature was raised to 0° C. after which lithium bis(trimethylsilyl)amide (1M solution; 14 mmol) was added in one portion and the mixture was stirred for 20 min at 0° C. and then at rt for 1 h. Hereafter ammoniumacetate (2.5 g; 14.1 mL) was added and the mixture was stirred for 60 min hereafter. The reaction was quenched with water/10% Na₂CO₃ (aq) and EtOAc. The organic phase was isolated, dried (MgSO₄) and evaporated to give a crude material (5.2 g) which was purified with flash chromatography, Isolera Isoctane/EtOAc 0% to 100% EtOAc to give 1.32 g pure title compound.

### 3-chloro-5,6,7,8-tetrahydro-1,6-naphthyridine (Intermediate Compound)

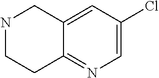

tert-Butyl 3-chloro-7,8-dihydro-5H-1,6-naphthyridine-6-carboxylate (0.16 g; 0.60 mmol) was dissolved in trifluoroacetic acid (1 mL) and stirred for 1 hr. The reaction mixture was evaporated in vacuo, then co-evaporated with MeOH and finally purified by ion exchange chromatography (MeOH 10 ml, then Et₃N/MeOH 1/4 50 ml) followed by evaporation giving a quantitative yield of title compound.

### 3-Chloro-6-[6-[(3R)-3-fluoropyrrolidin-1-yl]-2,4-dimethyl-5-nitro-1H-pyridin-2-yl]-7,8-dihydro-5H-1,6-naphthyridine (Intermediate Compound)

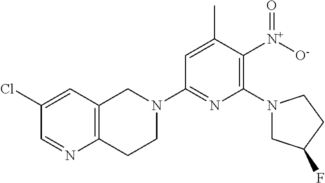

To a solution of 3-chloro-5,6,7,8-tetrahydro-1,6-naphthyridine (0.6 mmol) and 6-chloro-2-((R)-3-fluoro-pyrrolidin-1-yl)-4-methyl-3-nitro-pyridine (0.11 g; 0.42 mmol) in MeCN (10 mL) was added TEA (0.2 mL; 1.42 mmol) and heated at 60° C. over weekend. Hereafter the reaction mixture was evaporated to a total volume of 5 mL and transferred to micro oven vial, added additional TEA (0.2 mL) and heated in micro wave synthesizer at 100° C. for 3 hrs. The reaction mixture was evaporated to dryness and purified using flash chromatography (Isolera, 25 g cartridge, 5% EtOAc to 30% EtOAc) to yield 60 mg title compound (37%).

### 6-(3-chloro-7,8-dihydro-5H-1,6-naphthyridin-6-yl)-2-[(3R)-3-fluoropyrrolidin-1-yl]-4,6-dimethyl-1H-pyridin-3-amine (Intermediate Compound)

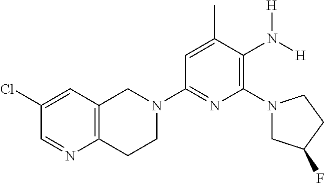

A slurry of 3-chloro-6-[6-[(3R)-3-fluoropyrrolidin-1-yl]-2,4-dimethyl-5-nitro-1H-pyridin-2-yl]-7,8-dihydro-5H-1,6-naphthyridine (30 mg; 0.08 mmol), ammoniumformate (0.097 mg; 1.53 mmol) and zinc (50 mg; 0.08 mmol) in 99% ethanol (2 mL) was heated at 70 C ° C. for 1.5 h. The reaction mixture was added EtOAc/10% Na₂CO₃, extracted, dried (MgSO₄) and evaporated in vacuo to yield 23 mg of the title compound. LC-MS 361/363 (M+1)

### Example 2

### N-[6-(3-chloro-7,8-dihydro-5H-1,6-naphthyridin-6-yl)-2-(3R)-3-fluoropyrrolidin-1-yl]-4,6-dimethyl-1H-pyridin-3-yl]-3,3-dimethyl-butanamide (Compound 2.1)

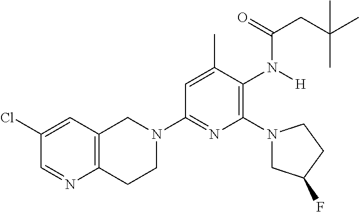

A solution of 6-(3-chloro-7,8-dihydro-5H-1,6-naphthyridin-6-yl)-2-[(3R)-3-fluoropyrrolidin-1-yl]-4,6-dimethyl-1H-pyridin-3-amine (23 mg) in THF (3 mL) is added tert-butylacetyl chloride (43 μL; 0.3 mmol) and stirred for 2 hrs. The title compound is purified as previously described (above) by flash coloumn chromatography. LC-MS 459/461 (M+1)

### Pharmacological Methods

### FLIPR-Based Characterization of Kᵥ7.2+3 Modulators

This experiment determines the ability of a test compound to modulate the activity of Kᵥ7.2+3 channels heterologously expressed in human HEK293 cells. The ability is determined relative to retigabine. The activity is determined using a standard thallium (I) sensitive assay, e.g. using a fluorometric method in a Fluorescent Image Plate Reader (FLIPR) as described below in more detail.

Full concentration/response curves are generated and EC₅₀ values are calculated based on max values. EC₅₀ values (Effective Concentration) represent the concentration of the test substance, at which 50% of the channel activity is obtained when compared to retigabine control responses. Maximal response determined relative to the reference (retigabine) response is calculated.

### Methods

### Cell Culture

Human HEK293 cells over-expressing human Kᵥ7.2+3 are grown in culture medium (DMEM supplemented with 10% foetal bovine serum), in polystyrene culture flasks (175 mm²) in a humidified atmosphere of 5% CO₂ in air, at 37° C. Cell confluence should be 80-90% on day of plating. Cells are rinsed with 4 ml of PBS (phosphate buffered saline) and incubated 2 min with 1 ml of Trypsin-EDTA. After addition of 25 ml of culture medium cells are re-suspended by trituration with a 25 ml pipette.

The cells are seeded at a density of ˜3×10⁶ cells/ml (25 μl/well) in blackwalled, clear bottom, 384-well plates pre-treated with 0.01 g/l poly-D-lysin (20 μl/well for ≧30 min). Plated cells were allowed to proliferate for 24 h before loading with dye.

Loading with BTC-AM

BTC-AM (50 mg, Invitrogen) is added 25.5 μl DMSO. The BTC-AM stock solution (2 mM) is diluted to a final concentration of 2 μM in Cl⁻ free assay buffer (in mM: 140 Na⁺-gluconate, 2.5 K⁺-gluconate, 6 Ca2⁺-gluconate, 1 Mg²⁺gluconate, 5 glucose, 10 HEPES, pH 7.3) containing 2 μM ouabain, 2 mM amaranth and 1 mM tartrazine.

The culture medium is aspirated from the wells, the cells are washed thrice in Cl⁻ free assay buffer, and 25 μl of the BTC-AM loading solution is added to each well. The cells are incubated at 37° C. for 60 min.

### TI⁺ Influx Measurements

After the loading period, the TI⁺-sensitive BTC fluorescence signal is measured over time using a FLIPR.

### FLIPR Settings/Parameters

Temperature: Room temp.
First addition: 12 μl test or control compound after 15 sec at a rate of 30 μl/sec and starting height of 20 μl
Second addition: 12 μl stimulus buffer (Cl⁻ free assay buffer supplemented with 1 mM Tl₂SO₄, 5 mM K₂SO₄ as well as the quenchers amaranth (2 mM) and tartrazine (1 mM)) is added after an additional 3 minutes at a rate of 30 μl/sec and starting height of 30 μl
Reading intervals: First sequence—3 sec×5, 2 sec×24 and 5 sec×25 Second sequence—1 sec×5, 2 sec×24 and 5 sec×36
   Addition plates (compound plate and stimulus plate) are placed in positions 2 and 3, respectively. Cell plates are placed in position 1 and run using the “KCNQ (two additions)” program. FLIPR will then take the appropriate measurements in accordance with the interval settings above. Fluorescence obtained after stimulation is corrected for the mean basal fluorescence (in Cl⁻ free assay buffer).

### Analysis

### Characterization of Active Substances

Full concentration/response curves are generated and EC₅₀ values (“Effective Concentration”; the concentration at which 50% of the channel activity is obtained when compared to retigabine control responses) are calculated based on peak values. Maximal response determined relative to the reference (retigabine) response is calculated. Data are given as mean of at least two test runs

TABEL 1

| | EC₅₀ | Efficacy |
|---|---|---|
| Test Compound | (μM) | (%) |
| 1.1 | 0.067 | 96 |
| 1.2 | 1.2 | 121 |
| 1.3 | 5.0 | 24 |
| 1.4 | 0.085 | 101 |
| 1.5 | 0.19 | 101 |
| 1.6 | 0.019 | 81 |
| 1.7 | 0.22 | 104 |
| 1.8 | 1.3 | 114 |
| 1.9 | 0.05 | 105 |
| 1.10 | 6.9 | 115 |
| 1.11 | 1.2 | 115 |
| 1.12 | 0.065 | 100 |
| 1.13 | 1.1 | 109 |
| 1.14 | 0.02 | 89 |
| 1.15 | 2.9 | 28 |
| 1.16 | 5.4 | 116 |
| 1.17 | 4.6 | 90 |
| 1.18 | 0.073 | 34 |
| 1.19 | 5.2 | 71 |
| 1.20 | 0.28 | 100 |

From the foregoing it will be appreciated that, although specific embodiments of the invention have been described herein for purposes of illustration, various modifications may be made without deviating from the spirit and scope of the invention. Accordingly, the invention is not to be limited as by the appended claims.

The features disclosed in the foregoing description, in the claims and/or in the accompanying drawings, may both separately and in any combination thereof, be material for realising the invention in diverse forms thereof.

### Preferred Features of the Invention:

1. A compound of Formula (I)

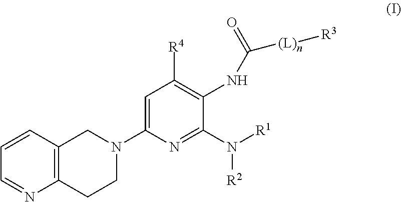

a stereoisomer or a mixture of its stereoisomers, or a pharmaceutically-acceptable addition salt thereof, or an N-oxide thereof, wherein
R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁₋₆-alkyl;
L represents a linker selected from —CR′R″—, —CH₂—CR′R″— and —CR′R″—CH₂—, wherein R′ and R″, independently of each other, represent hydrogen, C₁₋₆-alkyl or halo;
n is 0 or 1;
R³ represents C₁₋₆-alkyl, phenyl or furanyl, which phenyl and furanyl is optionally substituted one or more times with substituents selected from C₁₋₆-alkyl, C₁₋₆-alkoxy, halo and trifluoromethyl; and
R⁴ represents hydrogen, halo or C₁₋₆-alkyl.
2. The compound according to clause 1, a stereoisomer or a mixture of its stereoisomers, or a pharmaceutically-acceptable addition salt thereof, or an N-oxide thereof, wherein R¹ and R² together with the nitrogen to which they are attached is pyrrolidinyl, which pyrrolidinyl is optionally substituted one or more times with halo.
3. The compound according to any one of the clauses 1-2, a stereoisomer or a mixture of its stereoisomers, or a pharmaceutically-acceptable addition salt thereof, or an N-oxide thereof, wherein L represents —CH₂—.
4. The compound according to any one of the clauses 1-3, a stereoisomer or a mixture of its stereoisomers, or a pharmaceutically-acceptable addition salt thereof, or an N-oxide thereof, wherein R³ represents phenyl, which phenyl is optionally substituted one or more times with halo.
5. The compound according to any one of the clauses 1-3, a stereoisomer or a mixture of its stereoisomers, or a pharmaceutically-acceptable addition salt thereof, or an N-oxide thereof, wherein R³ represents C₁₋₆-alkyl.
6. The compound according to any one of the clauses 1-5, a stereoisomer or a mixture of its stereoisomers, or a pharmaceutically-acceptable addition salt thereof, or an N-oxide thereof, wherein R⁴ represents C₁₋₆-alkyl.
7. The compound according to clause 1, which is:
2-(3,5-Difluoro-phenyl)-N-[6-(7,8-dihydro-5H-[1,6]naphthyridin-6-yl)-4-methyl-2-pyrrolidin-1-yl-pyridin-3-yl]-acetamide;
N-[6-(7,8-Dihydro-5H-[1,6]naphthyridin-6-yl)-4-methyl-2-pyrrolidin-1-yl-pyridin-3-yl]-3,3-dimethyl-butyramide;
N-[6-(7,8-Dihydro-5H-[1,6]naphthyridin-6-yl)-4-methyl-2-pyrrolidin-1-yl-pyridin-3-yl]-3-fluoro-benzamide;
   or a stereoisomer or a mixture of its stereoisomers, or a pharmaceutically-acceptable addition salt thereof, or an N-oxide thereof.
   8. A pharmaceutical composition comprising a therapeutically effective amount of the compound according to any one of the clauses 1-7, a stereoisomer or a mixture of its stereoisomers, or a pharmaceutically-acceptable addition salt thereof, or an N-oxide thereof.
   9. Use of the compound according to any one of the clauses 1-7, a stereoisomer or a mixture of its stereoisomers, or a pharmaceutically-acceptable addition salt thereof, or an N-oxide thereof, for the manufacture of a pharmaceutical composition.
   10. Use of the compound according to any one of the clauses 1-7, a stereoisomer or a mixture of its stereoisomers, or a pharmaceutically-acceptable addition salt thereof, or an N-oxide thereof, for the manufacture of a pharmaceutical composition for the treatment, prevention or alleviation of a disease or a disorder or a condition of a mammal, including a human, which disease, disorder or condition is responsive to activation of KJ channels.
   11. The use according to clause 10, wherein the disease, disorder or condition is pain, neurodegenerative disorders, migraine, bipolar disorders, mania, epilepsy, convulsions, seizures and seizure disorders, anxiety, depression, schizophrenia and urinary incontinence.
   12. The use according to clause 10, wherein the disease, disorder or condition is pain, mild, moderate or severe pain, acute, chronic or recurrent pain, neuropathic pain, pain caused by migraine, postoperative pain, phantom limb pain, neuropathic pain, chronic headache, tension type headache, central pain, pain related to diabetic neuropathy, to post therapeutic neuralgia, or to peripheral nerve injury.
   13. A compound according to any of clauses 1-7, a stereoisomer or a mixture of its stereoisomers, or a pharmaceutically-acceptable addition salt thereof, or an N-oxide thereof, for use as a medicament.
   14. A compound according to any of clauses 1-7, a stereoisomer or a mixture of its stereoisomers, or a pharmaceutically-acceptable addition salt thereof, or an N-oxide thereof, for use in the treatment, prevention or alleviation of a disease or a disorder or a condition of a mammal, including a human, which disorder, disease or condition is responsive to activation of Kᵥ7 channels.
   15. A method of treatment, prevention or alleviation of a disease or a disorder or a condition of a living animal body, including a human, which disorder, disease or condition is responsive to activation of KJ channels, which method comprises the step of administering to such a living animal body in need thereof, a therapeutically effective amount of the compound according to any one of the clauses 1-7, a stereoisomer or a mixture of its stereoisomers, or a pharmaceutically-acceptable addition salt thereof, or an N-oxide thereof.

1. A compound of Formula (I) 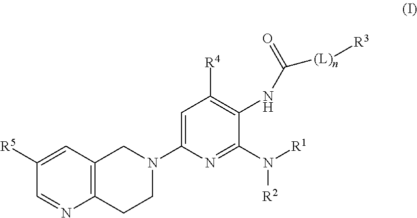
a stereoisomer or a mixture of its stereoisomers, or a pharmaceutically-acceptable addition salt thereof, or an N-oxide thereof, wherein
R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁-C₆-alkyl;
L represents a linker selected from —CR′R″—, —CH₂—CR′R″—, —CR′R″-CH₂—, and —O—, wherein R′ and R″, independently of each other, represent hydrogen, C₁₋₆-alkyl or halo;
n is 0 or 1;
R³ represents C₁₋₆-alkyl, phenyl, pyridyl, furanyl, imidazolyl, oxazolyl, isoxazolyl, thiazolyl, isothiazolyl, or C₃₋₆-cycloalkyl, which phenyl, pyridyl, furanyl, imidazolyl, oxazolyl, isoxazolyl, thiazolyl and isothiazolyl is optionally substituted one or more times with substituents selected from C₁₋₆-alkyl, C₃₋₆-cycloalkyl, phenyl, C₁₋₆-alkoxy, halo and trifluoromethyl;
R⁴ represents hydrogen, halo or C₁₋₆-alkyl; and
R⁵ represents hydrogen or halo.

2. The compound according to claim 1, a stereoisomer or a mixture of its stereoisomers, or a pharmaceutically-acceptable addition salt thereof, or an N-oxide thereof, wherein R¹ and R² together with the nitrogen to which they are attached is pyrrolidinyl or piperidinyl, which pyrrolidinyl and piperidinyl is optionally substituted one or more times with halo.

3. The compound according to claim 2, a stereoisomer or a mixture of its stereoisomers, or a pharmaceutically-acceptable addition salt thereof, or an N-oxide thereof, wherein R¹ and R² together with the nitrogen to which they are attached is pyrrolidinyl, which pyrrolidinyl is optionally substituted one or more times with halo.

4. The compound according to claim 1, a stereoisomer or a mixture of its stereoisomers, or a pharmaceutically-acceptable addition salt thereof, or an N-oxide thereof, wherein L represents —CH₂—.

5. The compound according to claim 1, a stereoisomer or a mixture of its stereoisomers, or a pharmaceutically-acceptable addition salt thereof, or an N-oxide thereof, wherein R³ represents C₁₋₆-alkyl.

6. The compound according to claim 1, a stereoisomer or a mixture of its stereoisomers, or a pharmaceutically-acceptable addition salt thereof, or an N-oxide thereof, wherein R³ represents phenyl, pyridyl, furanyl, imidazolyl, oxazolyl, isoxazolyl, thiazolyl, isothiazolyl, or C₃₋₆-cycloalkyl, which phenyl, pyridyl, furanyl, imidazolyl, oxazolyl, isoxazolyl, thiazolyl and isothiazolyl is optionally substituted one or more times with substituents selected from C₁₋₆-alkyl and halo.

7. The compound according to claim 1, a stereoisomer or a mixture of its stereoisomers, or a pharmaceutically-acceptable addition salt thereof, or an N-oxide thereof, wherein R⁴ represents C₁₋₆-alkyl.

8. The compound according to claim 1, a stereoisomer or a mixture of its stereoisomers, or a pharmaceutically-acceptable addition salt thereof, or an N-oxide thereof, wherein R⁵ represents hydrogen.

9. The compound according to claim 1, which is:
2-(3,5-Difluoro-phenyl)-N-[6-(7,8-dihydro-5H-[1,6]naphthyridin-6-yl)-4-methyl-2-pyrrolidin-1-yl-pyridin-3-yl]-acetamide
N-[6-(7,8-Dihydro-5H-[1,6]naphthyridin-6-yl)-4-methyl-2-pyrrolidin-1-yl-pyridin-3-yl]-3,3-dimethyl-butyramide;
N-[6-(7,8-Dihydro-5H-[1,6]naphthyridin-6-yl)-4-methyl-2-pyrrolidin-1-yl-pyridin-3-yl]-3-fluoro-benzamide;
or a stereoisomer or a mixture of its stereoisomers, or a pharmaceutically-acceptable addition salt thereof, or an N-oxide thereof.

10. The compound according to claim 1, which is:
N-[6-(7,8-dihydro-5H-1,6-naphthyridin-6-yl)-2-[(3R)-3-fluoropyrrolidin-1-yl]-4-methyl-3-pyridyl]-3-methyl-furan-2-carboxamide;
2-(3,5-difluorophenyl)-N-[6-(7,8-dihydro-5H-1,6-naphthyridin-6-yl)-2-[(3R)-3-fluoropyrrolidin-1-yl]-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-3-methyl-furan-2-carboxamide;
2-(3,5-difluorophenyl)-N-[2-(4,4-difluoro-1-piperidyl)-6-(7,8-dihydro-5H-1,6-naphthyridin-6-yl)-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-1-methyl-imidazole-2-carboxamide;
N-[2-(4,4-difluoro-1-piperidyl)-6-(7,8-dihydro-5H-1,6-naphthyridin-6-yl)-4-methyl-3-pyridyl]-5-methyl-oxazole-4-carboxamide;
N-[2-(4,4-difluoro-1-piperidyl)-6-(7,8-dihydro-5H-1,6-naphthyridin-6-yl)-4-methyl-3-pyridyl]-4-methyl-thiazole-5-carboxamide;
N-[2-(4,4-difluoro-1-piperidyl)-6-(7,8-dihydro-5H-1,6-naphthyridin-6-yl)-4-methyl-3-pyridyl]-3-methyl-isoxazole-4-carboxamide;
N-[2-(4,4-difluoro-1-piperidyl)-6-(7,8-dihydro-5H-1,6-naphthyridin-6-yl)-4-methyl-3-pyridyl]-4-methyl-oxazole-5-carboxamide;
2-cyclopropyl-N-[2-(4,4-difluoro-1-pipridyl)-6-(7,8-dihydro-5H-1,6-naphthyridin-6-yl)-4-methyl-3-pyridyl]acetamide;
N-[2-(4,4-difluoro-1-piperidyl)-6-(7,8-dihydro-5H-1,6-naphthyridin-6-yl)-4-methyl-3-pyridyl]-5-ethyl-oxazole-4-carboxamide;
N-[2-(4,4-difluoro-1-piperidyl)-6-(7,8-dihydro-5H-1,6-naphthyridin-6-yl)-4-methyl-3-pyridyl]-3-methyl-pyridine-2-carboxamide;
N-[2-(4,4-difluoro-1-piperidyl)-6-(7,8-dihydro-5H-1,6-naphthyridin-6-yl)-4-methyl-3-pyridyl]-2,5-dimethyl-oxazole-4-carboxamide;
N-[2-(4,4-difluoro-1-piperidyl)-6-(7,8-dihydro-5H-1,6-naphthyridin-6-yl)-4-methyl-3-pyridyl]-5-phenyl-oxazole-4-carboxamide;
5-cyclopropyl-N-[2-(4,4-difluoro-1-piperidyl)-6-(7,8-dihydro-5H-1,6-naphthyridin-6-yl)-4-methyl-3-pyridyl]oxazole-4-carb oxamide;
N-[2-(4,4-difluoro-1-piperidyl)-6-(7,8-dihydro-5H-1,6-naphthyridin-6-yl)-4-methyl-3-pyridyl]-5-methyl-isothiazole-4-carboxamide;
ethyl N-[2-(4,4-difluoro-1-piperidyl)-6-(7,8-dihydro-5H-1,6-naphthyridin-6-yl)-4-methyl-3-pyridyl]carbamate;
or a stereoisomer or a mixture of its stereoisomers, or a pharmaceutically-acceptable addition salt thereof, or an N-oxide thereof.

11. The compound according to claim 1, which is:
N-[6-(3-chloro-7,8-dihydro-5H-1,6-naphthyridin-6-yl)-2-[(3R)-3-fluoropyrrolidin-1-yl]-4,6-dimethyl-1H-pyridin-3-yl]-3,3-dimethyl-butanamide;
or a stereoisomer or a mixture of its stereoisomers, or a pharmaceutically-acceptable addition salt thereof, or an N-oxide thereof.

12. A pharmaceutical composition comprising a therapeutically effective amount of the compound according to claim 1, a stereoisomer or a mixture of its stereoisomers, or a pharmaceutically-acceptable addition salt thereof, or an N-oxide thereof.

13.-19. (canceled)

20. A method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the compound according to claim 1, a stereoisomer or a mixture of its stereoisomers, or a pharmaceutically-acceptable addition salt thereof, or an N-oxide thereof.

21. The method according to claim 20, wherein the disease, disorder or condition is pain, neurodegenerative disorders, migraine, bipolar disorders, mania, epilepsy, convulsions, seizures and seizure disorders, anxiety, depression, schizophrenia, essential tremors and urinary incontinence.

22. The method according to claim 21, wherein the disease, disorder or condition is pain, mild, moderate or severe pain, acute, chronic or recurrent pain, neuropathic pain, pain caused by migraine, postoperative pain, phantom limb pain, neuropathic pain, chronic headache, tension type headache, central pain, pain related to diabetic neuropathy, to post therapeutic neuralgia, or to peripheral nerve injury.
